(19) United States
(12) Patent Application Publication
(10) Pub. No.: US 2018/0094211 A1
(43) Pub. Date: Apr. 5, 2018

(54) MILD SURFACTANT FORMULATIONS FOR SKIN CLEANSING

(71) Applicant: RHODIA OPERATIONS, Paris (FR)

(72) Inventors: James GRIFFIN, Jackson, NJ (US); Rajesh PATEL, Pennington, NJ (US)

(73) Assignee: RHODIA OPERATIONS, Paris (FR)

(21) Appl. No.: 15/565,963

(22) PCT Filed: Apr. 13, 2016
(86) PCT No.: PCT/US16/27203

§ 371 Date: Oct. 12, 2017

Related U.S. Application Data
(60) Provisional application No. 62/146,494, filed on Apr. 13, 2015.

(51) Int. Cl. C11D 1/12; C11D 1/66; C11D 3/20; C11D 3/37; C11D 3/50; C11D 9/26; C11D 17/08; A61K 8/49; A61K 8/86; A61K 8/899; A61Q 19/10

(52) U.S. Cl. CPC C11D 1/123; C11D 1/667; C11D 3/2065; C11D 3/3707; C11D 3/50; A61Q 19/10; C11D 17/08; A61K 8/4993; A61K 8/86; A61K 8/899; C11D 9/26

(57) ABSTRACT

The present disclosure provides a cleansing composition that includes a combined amount of disodium alkyl ethoxy dimethicone sulfosuccinate; polyglycerol; and ethoxylated sorbitan ester.

### CROSS-REFERENCE TO RELATED APPLICATION

The present application claims the benefit of priority under 35 U.S.C. § 119(e) of U.S. Provisional Application Ser. No. 62/146,494, filed on Apr. 13, 2015, the entire disclosure of which is incorporated herein by reference.

### BACKGROUND

Consumers have in the past used absorbent substrates impregnated with topical compositions for a variety of purposes. For example, wipes impregnated with cleansing compositions are frequently used to conveniently wash hands and face while traveling or in public or anytime when water and soap are not readily accessible. Such wipes are also frequently used on babies to clean up during a diaper change.

As such products are typically applied to delicate skin, there is an ongoing need to develop mild, skin-friendly formulations.

### SUMMARY

The present disclosure provides a cleansing composition that includes a combined amount of disodium alkyl ethoxy dimethicone sulfosuccinate; polyglycerol; and ethoxylated sorbitan ester.

Also provided is a skin cleansing article that includes a substrate and a composition according to the present disclosure.

Also described is a skin cleansing method that includes the steps of applying a cleansing composition according to the present disclosure to an area of skin on a body; washing the area of skin on the body; and rinsing the area of skin on the body.

Also provided is a skin cleansing method that includes the step of contacting a skin cleansing article according to the present disclosure with an area of skin on a body.

### BRIEF DESCRIPTION OF THE DRAWINGS

FIG. 1 depicts a comparison of zein score of the formulation described in Example 1 (“Solvay”) compared to market products (“Market A-D”).

FIG. 2 depicts a comparison of results of foam analysis using DFA 100 foam analyzer.

FIG. 3 depicts viscosity of dilution path of Performance Concentrate.

### DETAILED DESCRIPTION

In general, cleansing compositions according to the present disclosure include a combined amount of disodium alkyl ethoxy dimethicone sulfosuccinate; a polyglycerol containing from 2 to 5 glycerol units; and ethoxylated sorbitan ester.

While specific embodiments are discussed, the specification is illustrative only and not restrictive. Many variations of this disclosure will become apparent to those skilled in the art upon review of this specification.

Unless defined otherwise, all technical and scientific terms used herein have the same meaning as is commonly understood by one of skill in the art to which this specification pertains.

As used in the specification and claims, the singular form “a”, “an” and “the” includes plural references unless the context clearly dictates otherwise.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Also, it should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

In certain embodiments, the disodium alkyl ethoxy dimethicone sulfosuccinate contains a C₁₀-C₁₈ alkyl group. In other embodiments, the disodium alkyl ethoxy dimethicone sulfosuccinate contains from 1 to 4 ethylene oxide units.

In certain embodiments, the polyglycerol contains from 2 to 5 glycerol units.

In other embodiments, the ethoxylated sorbitan ester is ethoxylated sorbitan monolaurate.

In certain embodiments, the cleansing composition includes a combined amount of disodium alkyl ethoxy dimethicone sulfosuccinate; a polyglycerol containing 3 glycerol units; and ethoxylated sorbitan monolaurate.

In certain embodiments, the composition further includes water. In some aspects, the water is present in an amount less than the combined amount of disodium alkyl ethoxy dimethicone sulfosuccinate; polyglycerol; and ethoxylated sorbitan ester. In another embodiment, the water is present in an amount greater than the combined amount of disodium alkyl ethoxy dimethicone sulfosuccinate; polyglycerol; and ethoxylated sorbitan ester.

The amounts of individual components in the cleansing composition will vary depending upon whether the composition is in a concentrated or diluted form. In certain embodiments, the disodium alkyl ethoxy dimethicone sulfosuccinate is present in an amount from about 20% to about 30% by weight based upon the total weight of the composition. In other embodiments, the disodium alkyl ethoxy dimethicone sulfosuccinate is present in an amount from about 0.5% to about 2% by weight based upon the total weight of the composition.

In certain embodiments, the polyglycerol is present in an amount from about 3% to about 9% by weight based upon the total weight of the composition. In other embodiments, the polyglycerol is present in an amount from about 0.1% to about 0.5% by weight based upon the total weight of the composition.

In certain embodiments, the ethoxylated sorbitan ester is present in an amount from about 6% to about 13% by weight based upon the total weight of the composition. In other embodiments, the ethoxylated sorbitan ester is present in an amount from about 0.2% to about 1% by weight based upon the total weight of the composition.

In the present disclosure, the term “solids” refers to all ingredients in a composition excluding water. In certain embodiments, the composition has a solids content ranging from 55% to 58%. When the composition has a high solids content, inclusion of preservatives is not required. Preservatives can be selected upon diluting the composition. In other embodiments, the composition has a solids content ranging from about 1.5% to about 3%.

In certain embodiments, the cleansing composition further includes one or more additives selected from vitamins, botanical extracts, moisturizers, emollients, preservatives, fragrances, and combinations thereof. In certain embodiments, the moisturizer is hydroxypropyl guar. Hydroxypropyl guar is multi-functional when used in the present compositions and articles. For example, it also provides a nice “slip” to the wipe during use. In other embodiments, hydroxypropyl guar modifies the surface of the wipe making it soft and cushiony.

Also provided is a skin cleansing article that includes a substrate and a composition according to the present disclosure. In certain embodiments, the composition includes a combined amount of disodium alkyl ethoxy dimethicone sulfosuccinate; a polyglycerol containing from 2 to 5 glycerol units; and ethoxylated sorbitan ester.

In certain embodiments, the substrate includes a water-insoluble material. In other embodiments, the water-insoluble material is selected from woven materials, nonwoven materials, foams, sponges, wadding, felts, balls, and films.

The present disclosure also provides a skin cleansing method that includes the steps of applying a composition according to the present disclosure to an area of skin on a body; washing the area of skin on the body; and rinsing the area of skin on the body. In certain embodiments, the composition is in the form of a make-up remover.

Also provided is a skin cleansing method that includes the step of contacting a skin cleansing article according to the present disclosure with an area of skin on a body. In certain embodiments, the article is in the form of a wipe, including but not limited to, baby wipes and wipes for removing make-up.

The present disclosure will further be described by reference to the following examples. The following examples are merely illustrative and are not intended to be limiting. Unless otherwise indicated, all percentages are by weight.

### EXAMPLE 1

### Preparation of Composition

A cleansing composition was prepared by combining the following components referenced in Table 1:

TABLE 1

| | % Active | Wt % | Active % |
|---|---|---|---|
| Formulation Component | Ingredient | in use | in use |
| DI Water | | 95.9 | |
| Sulfosuccinate (Mackanate ® Ultra SI) | 45.0 | 3.0 | 1.35 |
| Polyglycerol 3 | 100 | 0.2 | 0.2 |
| Ethoxylated sorbitan ester (Alkamuls ® | 100 | 0.3 | 0.3 |
| PSML 20) | | | |
| Na Benzoate | 100 | 0.4 | 0.4 |
| Hydroxypropyl Guar | 100 | 0.02 | 0.02 |

The formulation solids in the composition are 2.0%. In the present disclosure, the term “solids” refers to all ingredients in a composition excluding water.

### EXAMPLE 2

### Zein Analysis

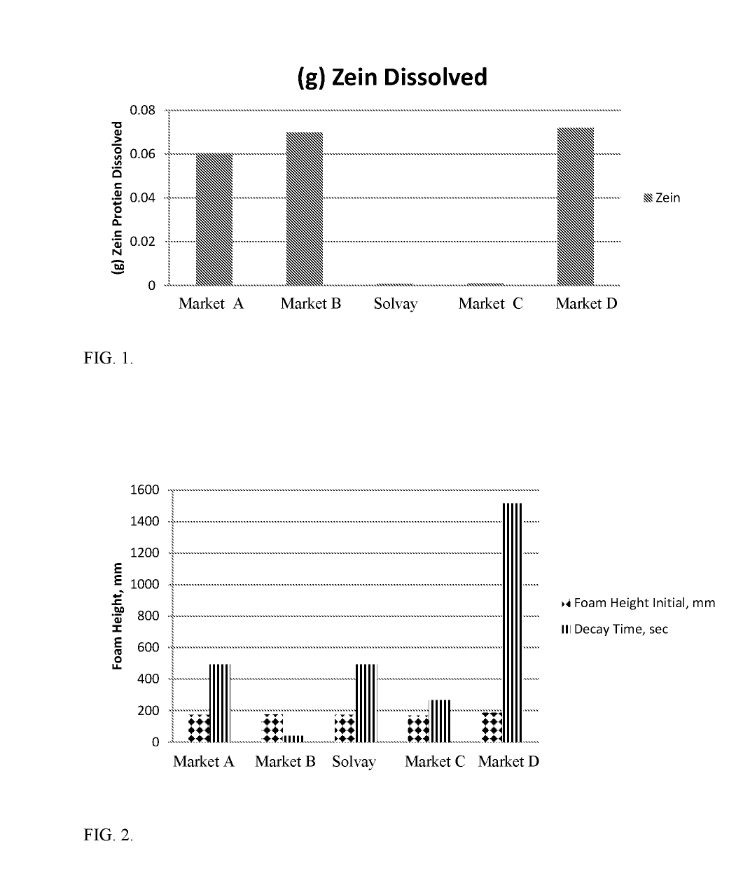

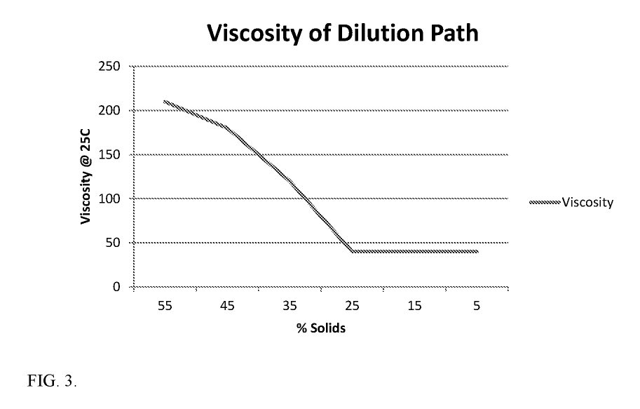

The Solvay formulation was one of the mildest compositions tested as shown by its low zein score in FIG. 1.

### EXAMPLE 3

### Foam Analysis

Foam height of all products was comparable. The range was found to be 168 -186 mm foam.

### EXAMPLE 4

### Concentrated Formulation

A concentrated formulation having a solids range of 54.0 -58.0% was prepared (“performance concentrate”) by combining the following components referenced in Table 2:

| Ingredient | wt % |
|---|---|
| Sulfosuccinate (Mackanate ® Ultra SI) | 85.71 |
| Polyglycerol 3 | 5.7 |
| Ethoxylated sorbitan ester (Alkamuls ® PSML 20) | 8.57 |

Because of the high solids content, inclusion of preservatives is not required. This performance concentrate is a clear, low viscosity liquid within the temperature range 4° C. to 45° C. The performance concentrate has a 28× dilution or let down level to make the wipe formulation. The freezing point is <0° C.

### Example 5

### Dilution of Concentrated Formulation

Samples were conditioned at various temperatures between 4° C. to 50° C. and viscosities recorded using a Brookfield Viscometer. The temperature range tested is essentially the storage and handling range of the product. The viscosity of the product is essentially the same between the temperature ranges of 20° to 50° C. Viscosities were measured at test temperature using Brookfield Viscometer LVT sp 3 @ 12 rpm.

### Example 6

### Stability Study

An accelerated aging study was conducted on the performance concentrate. Samples were aged at 4° C., 25° C., and 45° C. for 12 weeks. Three freeze thaw cycles were tested over −16° C. -25° C. One pH point of 5.9 was tested. The concentrated formulation was found to be stable in all conditions tested. As well as maintaining physical properties within the specification limits.

TABLE 3
Accelerated aging studies for performance concentrate.

| | Freeze/Thaw | | | |
|---|---|---|---|---|
| | Appear- | | | Appear- |
| | ance | Viscosity | pH as is | ance |
| | @ 25° C. | @ 25° C., cps | @ 25° C. | @ −16° C. |
| Cycle 1 | Pass | 210 | 5.91 | Solid |
| Cycle 2 | Pass | 210 | 5.94 | Solid |
| Cycle 3 | Pass | 210 | 5.88 | Solid |

| | Appear- | Viscosity | pH as | % Solids | |
|---|---|---|---|---|---|
| | ance | @ 25° C., | is @ | MW 40% | |
| | @ 25° C. | cps | 25° C. | 105° | Color |
| | 4° C. | | | | |
| 4 week | Pass | 210 | 5.93 | 55.7 | 1.2 |
| 8 week | Pass | 210 | 5.92 | 55.4 | 1.2 |
| 12 week | Pass | 210 | 5.86 | 55.6 | 1.2 |
| | 25° C. | | | | |
| 4 week | Pass | 210 | 5.86 | 55.6 | 1.1 |
| 8 week | Pass | 210 | 5.94 | 55.6 | 1.2 |
| 12 week | Pass | 210 | 5.98 | 55.8 | 1.2 |
| | 45° C. | | | | |
| 4 week | Pass | 210 | 5.96 | 55.9 | 1.3 |
| 8 week | Pass | 240 | 6.04 | 56.4 | 1.7 |

The disclosed subject matter has been described with reference to specific details of particular embodiments thereof. It is not intended that such details be regarded as limitations upon the scope of the disclosed subject matter except insofar as and to the extent that they are included in the accompanying claims.

Therefore, the exemplary embodiments described herein are well adapted to attain the ends and advantages mentioned as well as those that are inherent therein. The particular embodiments disclosed above are illustrative only, as the exemplary embodiments described herein may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the exemplary embodiments described herein. The exemplary embodiments described herein illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components, substances and steps. As used herein the term “consisting essentially of” shall be construed to mean including the listed components, substances or steps and such additional components, substances or steps which do not materially affect the basic and novel properties of the composition or method. In some embodiments, a composition in accordance with embodiments of the present disclosure that “consists essentially of” the recited components or substances does not include any additional components or substances that alter the basic and novel properties of the composition. If there is any conflict in the usages of a word or term in this specification and one or more patent or other documents that may be incorporated herein by reference, the definitions that are consistent with this specification should be adopted.

We claim:

1. A cleansing composition comprising a combined amount of disodium alkyl ethoxy dimethicone sulfosuccinate; polyglycerol containing from 2 to 5 glycerol units; and ethoxylated sorbitan monolaurate.

2. The cleansing composition of claim 1, wherein the composition further comprises water.

3. The cleansing composition of claim 2, wherein the water is present in an amount less than the combined amount of dimethicone sulfosuccinate; polyglycerol; and ethoxylated sorbitan monolaurate.

4. The cleansing composition of claim 2, wherein the water is present in an amount greater than the combined amount of dimethicone sulfosuccinate; polyglycerol; and ethoxylated sorbitan monolaurate.

5. The cleansing composition of claim 1, wherein the dimethicone sulfosuccinate is present in an amount from about 20% to about 30% by weight based upon the total weight of the composition.

6. The cleansing composition of claim 1, wherein the dimethicone sulfosuccinate is present in an amount from about 0.5% to about 2% by weight based upon the total weight of the composition.

7. The cleansing composition of claim 1, wherein the polyglycerol is present in an amount from about 3% to about 9% by weight based upon the total weight of the composition.

8. The cleansing composition of claim 1, wherein the polyglycerol is present in an amount from about 0.1% to about 0.5% by weight based upon the total weight of the composition.

9. The cleansing composition of claim 1, wherein the ethoxylated sorbitan monolaurate is present in an amount from about 6% to about 13% by weight based upon the total weight of the composition.

10. The cleansing composition of claim 1, wherein the ethoxylated sorbitan monolaurate is present in an amount from about 0.2% to about 1% by weight based upon the total weight of the composition.

11. The cleansing composition of claim 1, further comprising one or more additives selected from the group consisting of vitamins, botanical extracts, moisturizers, emollients, preservatives, fragrances, and combinations thereof

12. The cleansing composition of claim 1 further comprising a solids content of from about 1.5% to about 3%.

13. The cleansing composition of claim 1 further comprising a solids content of from about 55% to about 58%.

14. A skin cleansing article comprising a substrate and the composition of claim 1.

15. The skin cleansing article of claim 14, wherein the substrate comprises a water-insoluble material.

16. The skin cleansing article of claim 15, wherein the water-insoluble material is selected from the group consisting of woven materials, nonwoven materials, foams, sponges, wadding, felts, balls, and films.

17. A skin cleansing method comprising applying the cleansing composition of claim 1 to an area of skin on a body; washing the area of skin on the body; and rinsing the area of skin on the body.

18. A skin cleansing method comprising contacting the skin cleansing article of claim 14 with an area of skin on a body.
